(19) United States
(12) Patent Application Publication
(10) Pub. No.: US 2025/0161681 A1
(43) Pub. Date: May 22, 2025

(54) METHOD OF TREATING A PATIENT AND APPARATUS THEREFOR

(71) Applicant: MICHAEL MONTOYA, ONTARIO (CA)

(72) Inventor: MICHAEL MONTOYA, ONTARIO (CA)

(21) Appl. No.: 19/035,544

(22) Filed: Jan. 23, 2025

Related U.S. Application Data
(63) Continuation-in-part of application No. 18/521,757, filed on Nov. 28, 2023.
Application No. 18/521,757 is a continuation of application No. 16/803,843, filed on Feb. 27, 2020, now Pat. No. 11,826,566.
(60) Provisional application No. 62/821,213, filed on Mar. 20, 2019.

(51) Int. Cl. A61N 1/36; A61N 1/04

(52) U.S. Cl. CPC A61N 1/36034; A61N 1/0428; A61N 1/0452; A61N 1/0456; A61N 1/36003; A61N 1/36021

(57) ABSTRACT

A method of treating a patient includes an initial step of applying an initial scanning waveform to a patient's skin at a first area associated with a site of neurological disruption and monitoring to determine a first neurologically disrupted site. Upon locating at least one first neurologically disrupted site, the method can include scanning for at least a second neurologically disrupted site. Then the method may include conducting a frequency sweep to determine desired treatment frequencies, locating auxiliary treatment points, and treating the first and second neurologically disrupted sites concurrent with the auxiliary treatment points.

### CLAIM OF PRIORITY

The present application is a Continuation-In-Part (CIP) patent application of and claims priority to a previously filed, U.S. Non-Provisional patent application, namely, that having Ser. No. 18/521,757 and a filing date of Nov. 28, 2023, which is a Continuation patent application of and claims priority to a previously filed, U.S. Non-Provisional patent application, namely, that having Ser. No. 16/803,843, and a filing date of Feb. 27, 2020, which matured into U.S. U.S. Pat. No. 11,826,566 on Nov. 28, 2023; and further, claims priority to a previously filed, U.S. Provisional patent application, namely, that having Ser. No. 62/821,213 and a filing date of Mar. 20, 2019, with the contents of all prior applications being incorporated herein by reference in their entireties.

### BACKGROUND OF THE INVENTION

### Field of the Invention

The present invention relates to methods of treating a patient and apparatuses therefor, and more particularly to methods of introducing pulses to a patient through the use of an electrical neuromuscular stimulation apparatus for treatment of the patient. The methods result in the retraining of neuromuscular pathways to treat a variety of ailments.

### DESCRIPTION OF THE RELATED ART

It is well known that electrical energy, specifically pulses of electricity at various voltage levels and frequencies, can be used to heal and repair human tissue, can relieve undesirable and painful symptoms in a human, and even eliminate disease.

Under typical human movement, the brain sends impulses to the muscles via neurological pathways, thereby causing them to involuntarily contract. It has been found that stimulating the nervous system to contract the muscles using electrical pulses generally similar to the electrical impulses from the human's brain can have therapeutic effects, especially when combined with other therapies. With this in mind, various electrical stimulation units have been developed in order to provide therapies based on this principal. Accordingly, typical electrical muscle stimulation units tend to employ the use of pulses. The voltage level, frequency and duration of these pulses are based, to at least some degree, on the reaction and behavior of human muscle tissue to electrical impulses received from the brain.

It has been found that the human body responds to electrical pulses from electrical stimulation units increasing the volume blood and oxygen circulated to the area being treated. This increased circulation is the same natural process used by the body when performing a healing function. Accordingly, such electrical stimulation units are used for pain relief, decrease of inflammation, improved circulation, recovery from injury, fighting disease, muscle conditioning, and assisting muscles to contract properly.

The present invention provides significant advances in this field, and addresses several long-felt, but unresolved needs. By way of non-limiting example, the novel electronic circuitry of the present invention can employ DC coupled amplifiers to produce composite waveforms in the rage of 1 Hz to 50 KHz. Prior art apparatus generally rely on output transformers to generate waveforms, that, by their very nature can produce signals only in a narrow range.

### SUMMARY OF THE INVENTION

One aspect of the present invention includes the use of sophisticated and novel waveform generating apparatus, along with novel treatment methodologies, to implement a neuromuscular therapy that is vastly improved when compared to typical transcutaneous electrical nerve stimulation, neuromuscular electrical stimulation, and related therapies. One aspect of the novel treatment methodology employs the use of customized or specific waveforms for each patient and for each treatment area. The customized waveforms are determined by a frequency sweep of the waveform to determine an optimum waveform for the treatment. Repeated frequency scans May be employed as necessary, and enable the invention to tailor the treatment by identifying optimal neurological stimulating and neuromuscular waveforms that are specific to the patient's neurologically disrupted sites. The invention may also employ a symmetrical treatment configuration, in which the electrodes are mirrored on both sides of the patient's body, with one set of electrodes placed at the neurologically disrupted sites, and another set placed on unaffected tissue on the opposite side of the patient's body. This approach enhances overall balance in neuromuscular stimulation and contributes to holistic treatment outcomes.

In one embodiment of the present invention, a novel signal generating apparatus is employed to deliver waveforms to the tissue of the patient. The signal generating apparatus May include a waveform generation module, a neurological stimulation module, a frequency sweep mechanism, and a feedback mechanism, among other components. The waveform generation module is a component the generates a series of waveforms, including composite waveforms, ideally through a range that includes 1 Hz at the lower end and 50 KHz at the upper end. However, the inventor has considered that most use cases for the waveform generation module will only require a range of about 1 Hz to 30 KHz. The waveform generation module can be used to identify optimal treatment waveforms, such as an optimal neurological stimulating waveform. The neurological stimulation module sends multiple pulses once the desired treatment frequencies are determined. The frequency sweep mechanism allows an operator to scan various frequencies of the applied pulses in order to ascertain desired treatment frequencies. In a preferred embodiment, the mechanism is capable of sweeping through a range that includes 1 Hz and 50 KHz to identify the frequency that creates the least impedance within the patient's neurological tissue, thereby identifying a desired treatment frequency. The feedback mechanism may enable real time monitoring of the patient's responses to the applied treatment frequencies, and may also be configured to automatically and dynamically adjust certain parameters based on the patient's reaction.

In one embodiment, a treatment methodology employing the inventive apparatus begins with the application of a generic or standard initial waveform to the patient. It will be appreciated by those skilled in the art that the selection of waveform parameters may be determined by the treating technician or physician utilizing his or her judgment in light of the relevant circumstances, such as the particular treatment goals, physical aspects of the patient, and other relevant factors. In a preferred embodiment, a sinusoidal waveform in the range of about 1 Hz is applied, and is steadily increased to 30 KHz. The waveform parameters are monitored in order to determine an optimal neurological stimulating waveform. In a preferred embodiment, the optimal waveform will be the frequency at which impedance in the patient's tissue is minimized.

In alternative embodiments, the treatment methodology may involve the use of a composite waveform that includes at least two component signals. In such an embodiment, the first component of the waveform may be determined by applying a sinusoidal waveform in the range of about 1 Hz, and steadily increasing the waveform through to about 30 KHz. The optimal first component of the waveform will be the frequency at which impedance in the patient's tissue is minimized. To find the optimal second component of the waveform, a sinusoidal waveform in the range of about 1 Hz can be applied, composite with the first component, to the patient and steadily increased through to about 3000 Hz. The optimal second component will be that which results in maximum impedance in the patient's tissue when applied in composite with the first component.

Once an optimal waveform, or optimal composite waveform, is identified, the method May then call for scanning for sites of neurological disruption. This step may also call for the judgment of the treating technician or physician by applying waveforms to the affected area and monitoring the patient's response for a compensation pattern as the waveforms are applied to various areas within the treatment region. A compensation pattern may include any movement pattern adopted, consciously or unconsciously, to compensate for any kind of dysfunction or impairment in the human movement systems. As such, compensation patterns are typically thought of as “alternative” movement patterns as they may deviate from nominal or standard human movement patterns in order to provide compensation for the dysfunction or impairment. The patient may be guided to work the treatment area through various motions in order to facilitate the identification of a compensation pattern.

Once a first site of neurological disruption is located, in a preferred embodiment, a sequential scanning technique is employed to identify the next closest neurologically disrupted site. This may include applying the waveform moving progressively away from the first site in a spiral pattern. Once at least two sites of neurological disruption are identified, it may be necessary to repeat a frequency sweep to determine a desired treatment frequency and/or optimal neuromuscular waveform for the plurality of disrupted sites to be treated.

In a preferred embodiment, the treatment may consist of applying a waveform to the neurologically disrupted sites while instructing the patient to move the affected area through one or more predetermined routines. In a most preferred embodiment, the waveform is simultaneously applied to an unaffected area on the opposite side of the patient's body during treatment, concurrently treating both the neurologically disrupted area and the unaffected area. The simultaneous and symmetrical treatment method enhances overall balance in neuromuscular stimulation and contributes to holistic treatment outcomes.

In accordance with one aspect of the present invention there is disclosed yet another method of treating a patient, the method comprising the steps of generating a first series of pulses having a frequency in a range from about 1 Hz to about 3000 Hz, a voltage range from about 0 (zero) volts to about 100 (one hundred) volts, and a duty cycle of about 1% to about 90%; generating a second series of pulses having a frequency in a range from about 10 kHz to about 30 kHz, a voltage range from about 0 (zero) volts to about 100 (one hundred) volts, and a duty cycle of about 1% to about 90%; modulating the first series of pulses and the second series of pulses into a combined waveform of pulses; and delivering the combined waveform of pulses to the skin of the patient via an electrical circuit to thereby treat the patient.

In accordance with another aspect of the present invention there is disclosed a novel method of treating a patient, the method comprising the steps of generating a first series of pulses; generating a second series of pulses; modulating the first series of pulses and the second series of pulses into a combined waveform of pulses; and delivering the combined waveform of pulses to the skin of the patient via an electrical circuit to thereby treat the patient; monitoring the voltage and the current produced by the electrical circuit to attain a voltage value and a current value; calculating the impedance of the tissue of the patient using the voltage value and the current value; determining a voltage adjustment for at least one of the first series of pulses and the second series of pulses based on the impedance; adjusting the voltage of at least one of the first series of pulses and the second series of pulses based on the voltage adjustment to thereby control the output current level below a maximum threshold.

In accordance with another aspect of the present invention there is disclosed a novel method of treating a patient, the method comprising the steps of generating a first series of pulses; generating a second series of pulses; modulating the first series of pulses and the second series of pulses into a combined waveform of pulses; and delivering the combined waveform of pulses to the skin of the patient via an electrical circuit to thereby treat the patient; monitoring the voltage and the current produced by the electrical circuit to attain a voltage value and a current value; calculating the impedance of the tissue of the patient using the voltage value and the current value; displaying the calculated impedance.

In accordance with another aspect of the present invention there is disclosed a novel method of treating a patient, the method comprising the steps of generating a first series of pulses; generating a second series of pulses; modulating the first series of pulses and the second series of pulses into a combined waveform of pulses; and delivering the combined waveform of pulses to the skin of the patient via an electrical circuit to thereby treat the patient; monitoring the voltage and the current produced by the electrical circuit to attain a voltage value and a current value; calculating the impedance of the tissue of the patient using the voltage value and the current value; converting the calculated impedance to an impedance factor; and displaying the impedance factor.

In accordance with another aspect of the present invention there is disclosed a novel method of treating a patient, the method comprising the steps of generating a first series of pulses; generating a second series of pulses; modulating the first series of pulses and the second series of pulses into a combined waveform of pulses; and delivering the combined waveform of pulses to the skin of the patient via an electrical circuit to thereby treat the patient; monitoring the voltage and the current produced by the electrical circuit to attain a voltage value and a current value; calculating the impedance of the tissue of the patient using the voltage value and the current value; calculating an impedance value related to the calculated impedance; and displaying the impedance value and a benchmark impedance value for comparison purposes.

In accordance with another aspect of the present invention there is disclosed a novel method of treating a patient, the method comprising the steps of generating a first series of pulses; generating a second series of pulses; modulating the first series of pulses and the second series of pulses into a combined waveform of pulses; and delivering the combined waveform of pulses to the skin of the patient via an electrical circuit to thereby treat the patient; monitoring the voltage and the current produced by the electrical circuit to attain a voltage value and a current value; calculating the impedance of the tissue of the patient using the voltage value and the current value; calculating an impedance value related to the calculated impedance; calculating a comparison value based on the impedance value and a benchmark impedance value; and displaying the comparison value.

In accordance with another aspect of the present invention there is disclosed a novel method of treating a patient, the method comprising the steps of generating a first series of pulses; generating a second series of pulses; modulating the first series of pulses and the second series of pulses into a combined waveform of pulses; and delivering the combined waveform of pulses to the skin of the patient via an electrical circuit to thereby treat the patient; monitoring the voltage and the current produced by the electrical circuit to attain a voltage value and a current value; calculating the impedance of the tissue of the patient using the voltage value and the current value; calculating an impedance value related to the calculated impedance; determining an action to be taken based on the impedance value and a benchmark impedance value; and displaying the action to be taken.

In accordance with another aspect of the present invention there is disclosed a novel method of treating a patient, the method comprising the steps of generating a first series of pulses; generating a second series of pulses; modulating the first series of pulses and the second series of pulses into a combined waveform of pulses; and delivering the combined waveform of pulses to the skin of the patient via an electrical circuit to thereby treat the patient; monitoring the voltage and the current produced by the electrical circuit to attain a voltage value and a current value; calculating the impedance of the tissue of the patient using the voltage value and the current value; calculating an impedance value related to the calculated impedance; automatically adjusting the first series of pulses and the second series of pulses based on the impedance value.

In accordance with another aspect of the present invention there is disclosed a novel method of treating a patient, the method comprising the steps of generating a first series of pulses; generating a second series of pulses; modulating the first series of pulses and the second series of pulses into a combined waveform of pulses; and delivering the combined waveform of pulses to the skin of the patient via an electrical circuit to thereby treat the patient; monitoring the voltage and the current produced by the electrical circuit to attain a voltage value and a current value; calculating the impedance of the tissue of the patient using the voltage value and the current value; calculating an impedance value related to the calculated impedance; determining an action to be taken based on the impedance value and a benchmark impedance value; and automatically adjusting the first series of pulses and the second series of pulses based on the action to be taken.

In accordance with another aspect of the present invention there is disclosed a novel method of treating a patient, the method comprising the steps of generating a first waveform and applying the first waveform to the skin of the patient via an electrode; monitoring the impedance of the waveform within the patient's tissue; sweeping the frequency of the waveform between 10 KHz and 30 KHz; identifying a frequency at which the impedance is at a maximum; generating a second waveform and applying the waveform to the skin of the patient via an electrode; sweeping the frequency of the second waveform between 1 Hz and 3000 Hz; identifying a frequency at which the impedance is at a maximum; and treating the patient.

Yet another feature of the present invention may also include the application of waveforms via electromagnetic fields. While waveforms may be most efficiently applied to a patient via electrodes, an electromagnetic coil may also be used in lieu of, or in addition to, the aforementioned electrodes. An electromagnetic field generated by a coil may enhance neuromuscular stimulation by assisting the flow of electrons through the other channels (electrodes). This collaborative effect contributes to the generation of electrical currents through the patient's tissue. Manual application of pulsed electromagnetic fields can also provide therapeutic affects in conjunction with the present invention, which may operate by opening both the cardiovascular and neurological systems in the affected area, resulting in improved blood circulation and neurological responsiveness.

Other advantages, features and characteristics of the present invention, as well as methods of operation and functions of the related elements of the structure, and the combination of parts and economies of production, will become more apparent upon consideration of the following detailed description and the appended claims with reference to the accompanying drawings, the latter of which is briefly described herein below.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a side elevational view of the apparatus according to the present invention;

FIG. 2 is a cut-away perspective view of the apparatus of FIG. 1;

FIG. 3 is an electrical schematic that is representative of the apparatus and method according to the present invention of FIG. 1, and showing a representation of a combined waveform of pulses used to treat a patient;

FIG. 4 is a scan on an oscilloscope showing the first series of pulses and the second series of pulses;

FIG. 5 is a front plan view of a cellular telephone connected in data transfer relation to the apparatus of FIG. 1, and displaying a calculated impedance, an impedance factor, an impedance value, a benchmark impedance value, a comparison value and an action to be taken;

FIG. 6 is a perspective view from the side of the apparatus according to the present invention connected to a patient for providing treatment, and with the patient holding the horizontal stability post;

FIG. 7 is an enlarged top plan view of a portion of the rotary dial mounted on the horizontal stability post, and showing the patient using one hand to adjust the rotary dial;

FIG. 8 is a flow chart demonstrating one method of treating a patient according to one embodiment of the present invention; and

FIG. 9 is a flow chart demonstrating one method of sweeping frequencies to determine desired frequencies according to one embodiment of the present invention.

Like reference numerals refer to like parts throughout the several views of the drawings.

### DETAILED DESCRIPTION OF THE PREFERRED EMBODIMENT

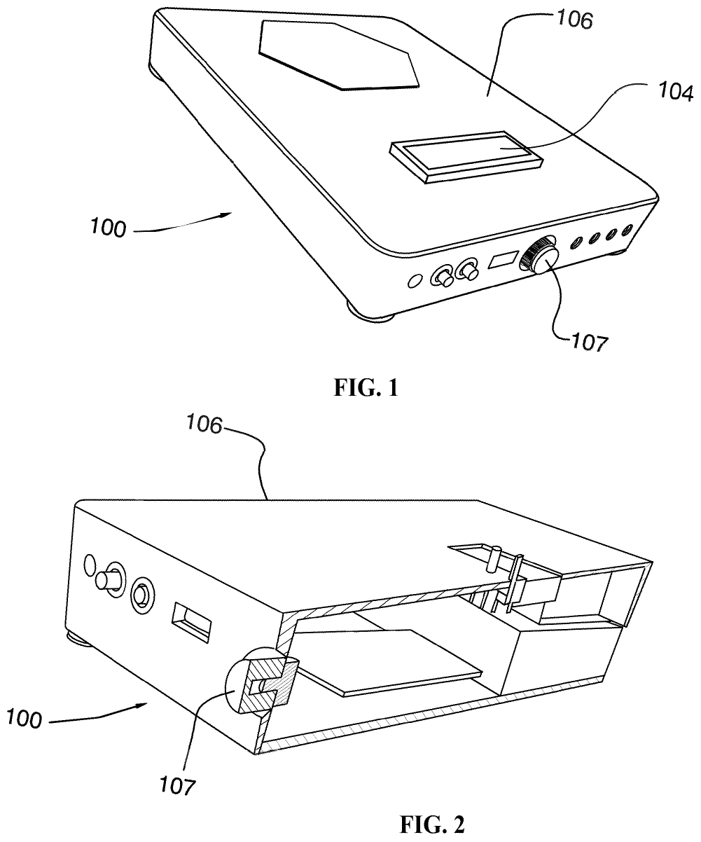

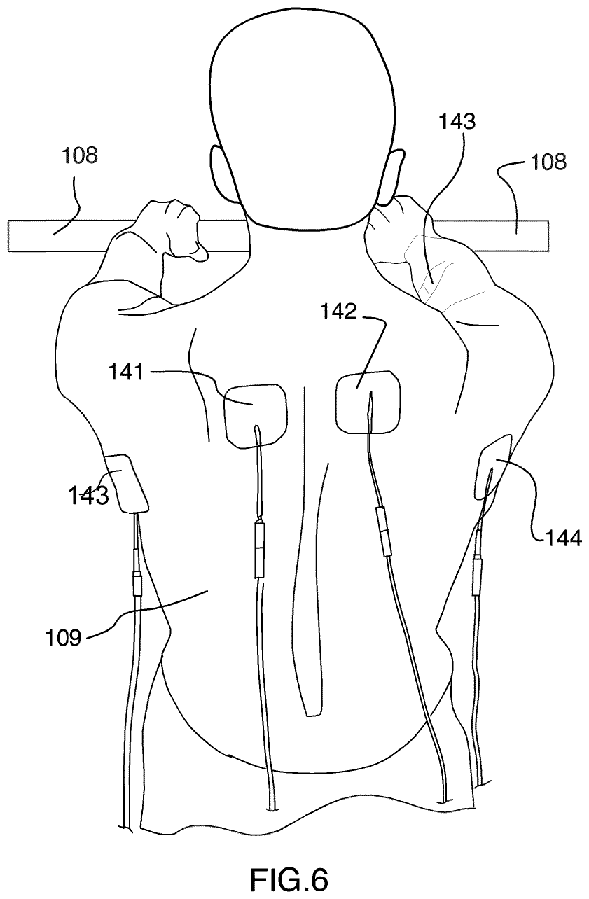

Reference will now be made to FIGS. 1 through 7, which show an illustrated embodiment of the method of treating a patient and apparatus therefor according to the present invention.

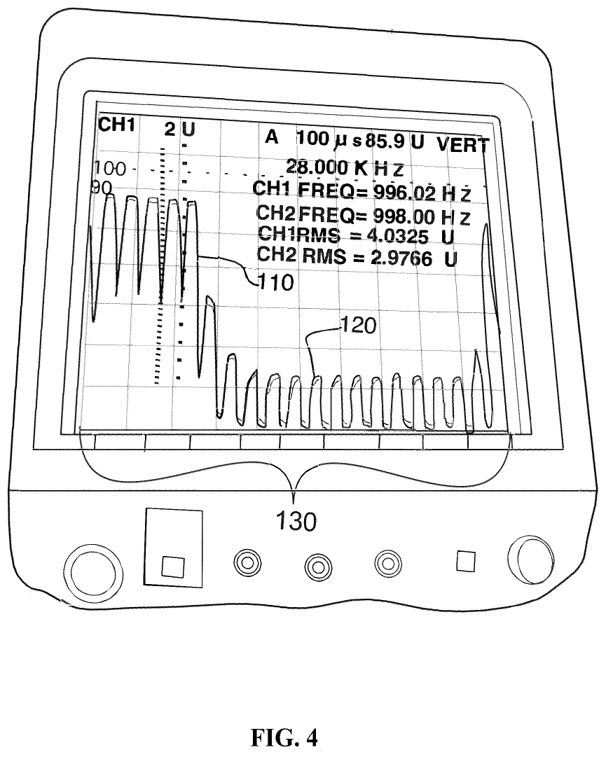

In one aspect, the present invention comprises a novel method of treating a patient 109. The method comprises the steps of generating a first series of pulses 110 and generating a second series of pulses 120. In the present invention, a signal generating apparatus 100 is used. In the disclosed embodiment, the first series of pulses 110 has a frequency in a range from about 1 Hz to about 5 kHz, a voltage range from about 0 (zero) volts to about 100 (one hundred) volts, and a duty cycle of about 1 to about 90%. The second series of pulses 120 has a frequency in a range from about 5 kHz to about 50 kHz, a voltage range from about 0 (zero) volts to about 100 (one hundred) volts, and a duty cycle of about 1% to about 90%. The method also includes the step of modulating the first series of pulses 110 and the second series of pulses 120 into a combined waveform of pulses 130, as shown in FIG. 4. Subsequently to modulating the first series of pulses and the second series of pulses 120, the combined waveform of pulses 130 is delivered to the skin of the patient 109 via an electronic circuit to thereby treat the patient 109. In the disclosed embodiment, the electronic circuit may comprise a first pair of electrodes (first electrode pad 141 and second electrode pad 142) and a second pair of electrodes (third electrode pad 143 and fourth electrode pad 144). The first pair of electrodes is attached in electrically conductive relation to a patient's skin at a first area. Similarly, the second pair of electrodes is attached in electrically conductive relation to a patient's skin at a second area. The output, or in other words the combined waveform of pulses 130, from the first pair of electrodes is the same as the output from the second pair of electrodes. Further, the output, or in other words the combined waveform of pulses 130, from the first electrode pad 141 is typically the same waveform as the output from the third electrode pad 143, and the output, or in other words the combined waveform of pulses 130, from the second electrode pad 142 is typically the same waveform as the output from the fourth electrode pad 144; however, it is common to have the amplitude different one pair of electrodes from the other.

The depicted embodiments also include the step of monitoring the voltage and the current produced by the electronic circuit to attain a voltage value and a current value. The voltage and current values that are produced by the electronic circuit 102 of the apparatus 100 according to the present invention are measured and used by the electronic circuit 102 itself via feedback loop current circuit 170. Further, the voltage and current values are measured at a point in time, and may be measured as frequently as desired. Also, the voltage and current values are measured with respect to first electrode pad 141 and the second electrode pad 142, and also are measured between the third electrode pad 143 and the fourth electrode pad 144, to thereby produce the measured voltage and current values.

Subsequent to measuring the voltage values and current values, the step of calculating the impedance of the tissue of the patient 109 using the voltage value and the current value is performed. The calculated impedance is indicative of the impedance, or other words resistance, to electrical flow of the tissue of the patient 109 between the electrodes.

The next step is that of determining a voltage adjustment for at least one of the first series of pulses 110 and the second series of pulses 120 based on the impedance. Since the first series of pulses 110 and the second series of pulses 120 have been modulated together to form a combined waveform of pulses 130, the measurement of is the maximum voltage of the combined waveform. It has been found that the frequency may be adjusted within a range from about 1 Hz to about 5 kHz, the voltage may be adjusted within a range from about 0 (zero) volts to about 100 (one hundred) volts, and the duty cycle may be adjusted within a range of about 1% to about 90%. The voltage is measured peak-to-peak and therefore ranges from +50 volts to −50 volts).

If desired, the next step may be that of automatically adjusting the voltage of at least one of the first series of pulses 110 and the second series of pulses 120 based on the voltage adjustment that has been determined. Such automatic adjustment of the voltage of at least one of the first series of pulses 110 and the second series of pulses 120 is performed to thereby control the output current level below a maximum threshold. In the present invention, the step of monitoring the voltage across the pair of electrodes and the current through the pair of electrodes is performed at a time interval of about between about 10 ms and 100 ms, and even more specifically is performed at a time interval of about 50 ms.

Further, the step of adjusting the voltage of at least one of the first series of pulses 110 and the second series of pulses 120 according to the calculated impedance to thereby control the output current level below a maximum threshold may comprise adjusting the voltage of the first series of pulses 110 according to the calculated impedance to thereby control the output current level below a maximum threshold. Alternatively, or additionally, the step of adjusting the voltage of at least one of the first series of pulses 110 and the second series of pulses 120 according to the calculated impedance to thereby control the output current level below a maximum threshold may comprise adjusting the voltage of the second series of pulses 120 according to the calculated impedance to thereby control the output current level below a maximum threshold. Also, the step of adjusting the voltage of at least one of the first series of pulses 110 and the second series of pulses 120 according to the calculated impedance to thereby control the output current level below a maximum threshold may comprise adjusting the voltage of both the first series of pulses 110 and the second series of pulses 120 according to the calculated impedance to thereby control the output current level below a maximum threshold.

It has been determined through experimentation that the step of adjusting the voltage of at least one of the first series of pulses 110 and the second series of pulses 120 according to the calculated impedance to thereby control the output current level below a maximum threshold May advantageously comprise reducing the voltage by about 5% of at least one of the first series of pulses 110 and the second series of pulses 120 according to the calculated impedance to thereby control the output current level below a maximum threshold.

It is further contemplated that the step of adjusting the frequency of the first series of pulses may comprise adjusting the frequency of one or both of the first series of pulses 110 and the second series of pulses 120 according to the calculated impedance.

It has also been found that in the present invention, communicating the results of the impedance values that are determined by the measurements of the voltage and current at the output of the electronic circuit according to the present invention is of significance. Accordingly, an important step in the method of the present invention comprises displaying the calculated impedance 151 a, such as on a digital display 104 on the apparatus 100. The calculated impedance is expressed in ohms and is derived by dividing the voltage by the current. A person knowledgeable in this art, or generally knowledgeable in the art of electronics, may be comfortable with dealing with a displayed impedance value expressed in ohms; however, in the present invention, the method according to the present invention may further comprise the step of converting the calculated impedance to an impedance factor 151 b. Further, there may be the step of displaying the impedance factor 151 b. The impedance factor 151 b may be expressed on a scale such as a cardinal scale of 1 (one) to 10 (ten), or similar, or any other convenient scale or the like that would be meaningful to a technician or a patient 109.

Further, a maximum impedance factor and/or a minimum impedance factor may be displayed. The maximum impedance factor might represent the maximum desired impedance of the muscle tissue and the minimum impedance factor might represent the minimum desired impedance of the muscle tissue. Encountering measured impedance factors outside of the range of the maximum impedance factor and the minimum impedance factor can indicate potential problems with the cells of the muscle tissue.

A desired impedance factor might also be displayed. The desired impedance factor could be a guide to perhaps an ideal physiological condition of the cells of the muscle tissue being treated, and could be used as a guide as to whether the treatment is helping the cells with the muscle tissue.

A target impedance factor might also be displayed. The target impedance factor could be a temporary target value or a final targeted value that is trying to be reached given the type of muscle tissue being treated and information about the possible injuring or illness.

A standardized impedance factor might also be displayed. The standardized impedance factor could be an impedance factor that is accepted in the physiological treatment profession as being a value that my general would be expected for the particular treatment in that particular area of the human body, possibly also considering given the conditions that are being encountered.

Further, the present invention might include the steps of calculating an impedance value 151 c related to the calculated impedance, and also displaying the impedance value 151 c and a benchmark impedance value for comparison purposes. The impedance value and a benchmark impedance value 151 d might be expressed in physiologically related terms, such as oxygen saturation in cells, protein levels in cells, and so on. A benchmark impedance value 151 d is May be defined as a standard of excellence, achievement, and so on, against which similar things May be measured or judged.

Additionally, the steps of calculating a comparison value 151 e based on the impedance value and the benchmark impedance value 151 d, and displaying the comparison value could be performed in order to qualitatively and/or quantitatively relate the impedance value and the benchmark impedance value in a manner that is meaningful and can be readily understood by a technician, a patient 109, or the like.

Also, the steps of determining an action to be taken 151 f based on the impedance value and the benchmark impedance value, and displaying the action to be taken 151 f can be performed. The displayed action may comprises reducing or increasing voltage of at least one of the first series of pulses 110 and the second series of pulses 120. Alternatively, or additionally, the steps of calculating an impedance value related to the calculated impedance and automatically adjusting the first series of pulses 110 and the second series of pulses 120 based on the impedance value could be performed in order to provide automatic adjustment of the pulse is provided to the patient 109, thereby helping to optimize the treatment. The step of automatically adjusting the first series of pulses 110 and the second series of pulses 120 based on the impedance value could comprise automatically adjusting at least one of the frequencies, the duty cycle and the voltage of the first series of pulses 110 and the second series of pulses 120. Further an electrical current value within a desired range or within a pre-determined range could be selected.

Also, or additionally, the present invention could include the steps of calculating an impedance value related to the calculated impedance and determining an action to be taken based on the impedance value and a benchmark impedance value, and automatically adjusting the first series of pulses 110 and the second series of pulses 120 based on the action to be taken. For instance, the action to be taken could be to decrease the voltage by 10% and slowly increase the voltage incrementally to see what resulting impedance is produced.

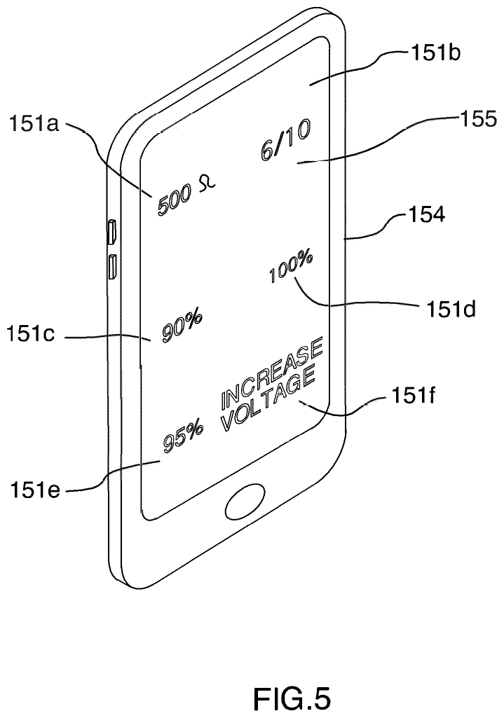

Reference will now be made to FIGS. 5 through 7, which show the apparatus according to the present invention in use. The first electrode pad 141, the second electrode pad 142, the third electrode pad 143 and the fourth electrode pad 144 are secured in electrically conductive relation to the patient's skin. Also, a horizontal stability post 108 is gripped by the patient 109 in order to help the patient 109 have proper upright posture that is literally even, and also to indicate when the patient's posture might become improper or uneven. The apparatus can readily be used to properly treat the patient 109 according to the method of the present invention.

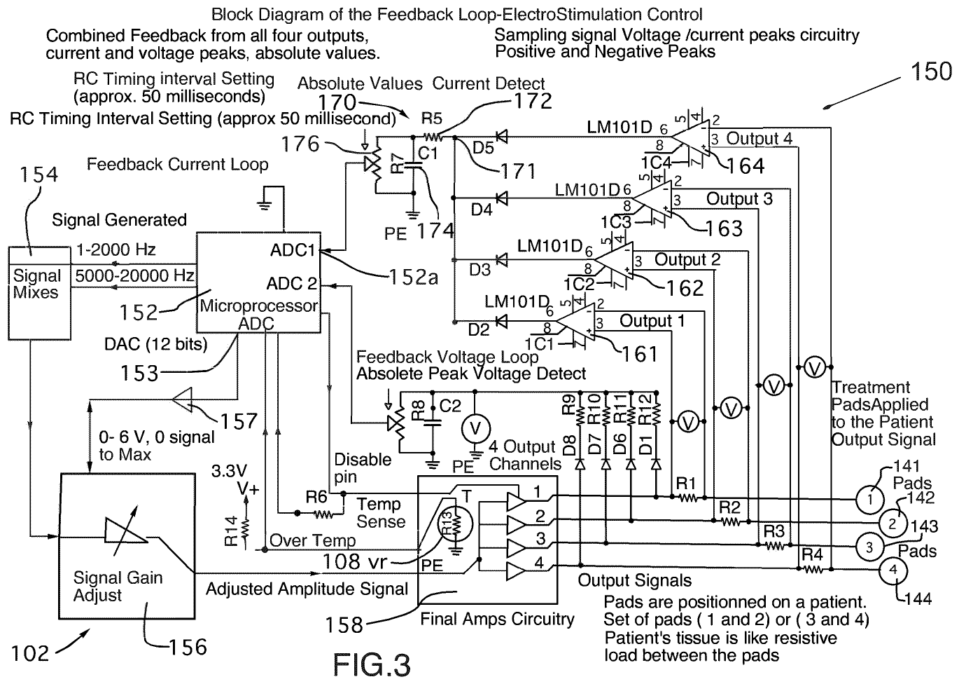

Reference will now be made to FIG. 3, which shows the electrical schematic that is representative of the circuit 150 that is the basis of the apparatus 100 and method according to the present invention. In a preferred embodiment, the microprocessor 152 that is used is a STM32 produced by ST Microelectronics, part number F303K8, however any suitable microprocessor 152 could alternatively be used. The microprocessor 152 is programmed to generate the first series of pulses 110 and the second series of pulses 120 that have the various voltage, frequency, and duty cycle characteristics as set forth above.

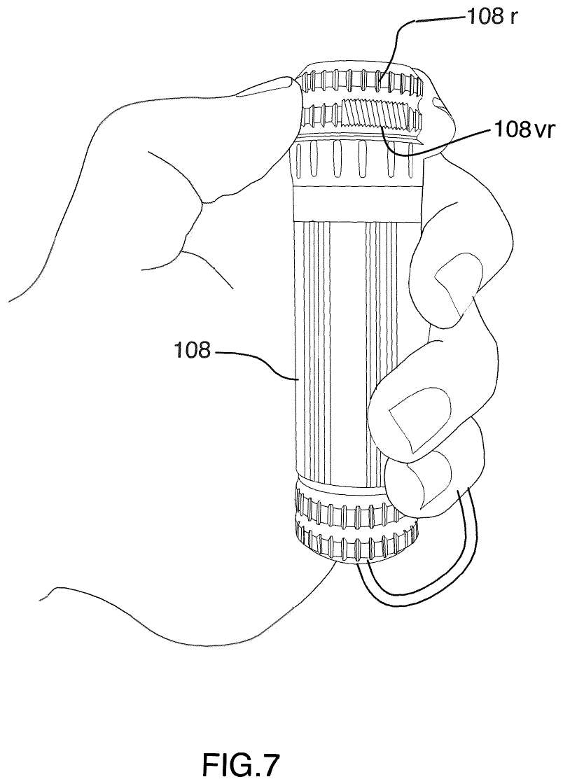

The first series of pulses 110 and the second series of pulses 120 are modulated in the mixer circuitry 154 to thereby form the combined waveform of pulses 130, which is fed into a signal gain adjust circuit 156. In a preferred embodiment, the signal gain adjust circuit 156 includes a compander or expander, and most preferably includes a linear gain compander circuit. It has been determined that a linear gain compander or expander circuit provides exceedingly fine control of the amplitude of the combined waveform of pulses 130. The compander circuit may include a compressor at the input side and an expander on the output side, such that the signal is first compressed upon entering the signal gain adjust circuit 156, thereby reducing its dynamic range. The gain is then adjusted by modulating the compressed signal. Before leaving the signal gain adjust circuit 156, the signal is then expanded back to its original dynamic range. The signal gain adjust circuit 156 is used to permit selective adjustment of the voltage level of the first series of pulses 110 and the second series of pulses 120. This adjustment can be made by a technician or by the patient 109. In the illustrated embodiment of FIGS. 1 and 2, this adjustment can be made by turning the adjustment knob 107 on the main housing 106 of the apparatus 100 according to the present invention. Alternatively, as can be seen in FIG. 7, a rotary dial 108 r of one end of the horizontal stability post 108 turns a variable resistor 108 vr, or the like. The variable resistor is connected in electrically conductive relation to the signal gain adjust circuit 156 to allow for adjustment of the voltage level of the first series of pulses 110 and the second series of pulses 120 of the combined waveform of pulses 130 via the rotary dial.

The microprocessor 152 also feeds a DC voltage through a doubling amplifier 157 into the signal gain adjust circuit 156 in order to control the amplitude of the output waveform. The output of the signal gain adjust circuit 156 is fed into a final amplifier 158 having four output channels. Each of the four output channels is fed through a separate resistor (R1,R2,R3,R4) to a corresponding electrode pad, namely first electrode pad 141, a second electrode pad 142, a third electrode pad 143, and a fourth electrode pad 144. The four electrode pads are each placed securely in electrically conductive relation to the skin of a patient 109 at the area to be treated. The combined waveform of pulses 130 created from the first series of pulses 110 and the second series of pulses is fed through the four electrode pads. The combined waveform of pulses 130 is present across the first electrode pad 141 and second electrode pad 142, and similarly is present across the third electrode pad 143 and fourth electrode pad 144.

As is discussed above, the impedance of the tissue of the patient 109 between the first electrode pad 141 and the second electrode pad 142 and also between the third electrode pad 143 and fourth electrode pad 144 voltage by the current can be calculated by dividing the voltage by the current. The voltage output of the first electrode pad 141 and the second electrode pad 142 is determined. Similarly, the voltage output of the third electrode pad 143 and the fourth electrode pad 144 is determined.

More specifically, at each of the four outputs of the final amplifier 158, there is a resistor (R1,R2,R3,R4) that, in the depicted embodiment, has a value of ten (10) ohms. The voltage across R1 leading to the first electrode pad 141 is fed back into the inputs of a first operational amplifier 161. Similarly, the voltage across R2 leading to the second electrode pad 142 is fed back into the inputs of a second operational amplifier 162, the voltage across R3 leading to the third electrode pad 143 is fed back into the inputs of a third operational amplifier 163, and the voltage across R4 leading to the fourth electrode pad 144 is fed back into the inputs of a fourth operational amplifier 164. The outputs of the four operational amplifiers are fed through diodes to a common input 171 of a feedback loop current circuit 170, specifically through a resistor 172, to act on the capacitor 174. The voltage across capacitor 174 changes as the voltages from the operational amplifiers change. In essence, the resistor 172 and the capacitor 174 act to “smooth out” the peak voltages so that extreme variations of the voltage do not affect the feedback operation of the overall electronic circuit 102.

The voltage across the capacitor 174 is applied as an absolute value into a first input 152a of the microprocessor 152. If the voltage received by the first input 152a of the microprocessor 152 increases greater than a threshold amount, the microprocessor 152 decreases the voltage of the first series of pulses 110 and/or the second series of pulses 120 by about 5%. The variable resistor 176 is used to calibrate the feedback loop current circuit 170.

The feedback loop current circuit 170 detects absolute value current peaks and absolute value peak voltage peaks of each of the first electrode pad 141, the second electrode pad 142, the third electrode pad 143, and the fourth electrode pad 144. The feedback loop current circuit 170 feeds the voltage values to the microprocessor 152 through pins ADC1 and ADC2. The feedback values are read by the microprocessor 152 at intervals as programmed into the microprocessor 152. The voltage is held by the feedback loop current circuit 170 until read by the microprocessor 152.

As can be readily seen, the electronic circuit 102 according to the depicted embodiment employs DC coupled amplifiers to thereby faithfully produce an output that is a combined waveform of pulses. The DC coupled amplifiers accurately and faithfully produce all of the pulse frequencies including 1 Khz to 50 Khz.

The microprocessor 152 evaluates the input absolute values from the feedback loop current circuit 170. If the peak current equals or surpasses the peak limit of 100 mA, the microprocessor 152 is instructed to reduce the DAC value output. In a most preferred embodiment, 100 mA is the regulated maximum safety level current value. The DAC output pin 153 (12 bits) controls the mixed signal wave voltage amplitude level, from zero to a maximum signal. The DAC values are referenced by voltage levels, between 0 and 6 volts.

The microprocessor 152 may also send message to the APP controller 155 running on a cellular telephone 154 (FIG. 5), or a portable tablet type computer, to limit the upper values being sent to the device, and prompt the user to scale back the sent values.

The microprocessor 152 can also disable the final amplifier 158 if there is a safety condition unmet, depending on the feedback conditions, including temperature sensing.

The microprocessor 152 can also monitor the calibration limits of the electronic circuit 102. A message may also be sent to the APP controller 155 running on a cellular telephone 154, if the electronic circuit 102 is out of calibration. The electronic circuit 102 as illustrated has the following calibration limit conditions:

$$25 \% of Amplitude = V 25 = X value + / - 10 \%$$

$$50 \% of Amplitude = V 50 = Y value + / - 10 \%$$

$$75 \% of Amplitude = V 75 = Z value + / - 10 \%$$

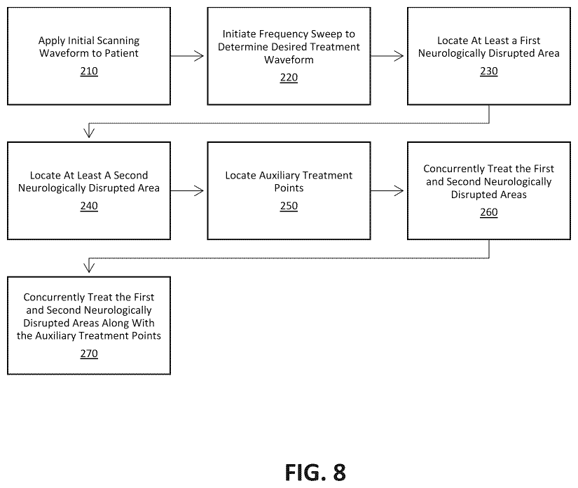

Now turning to FIG. 8, one embodiment of a treatment methodology according to the present invention is disclosed. As an initial step, the method may call for the application of an initial scanning waveform to the patient 210 using an electrode. The parameters of the waveform may be determined by the operator using his or her judgment in light of the circumstances, but nominally may be a sinusoidal waveform having a frequency of about 1 Hz and a voltage of between 0 and 100 volts. The waveform may also be any other suitable form, such as square, sawtooth, or pulse-width modulated. The initial scanning waveform may also comprise a composite waveform, utilizing a combination of two or more frequencies.

Next, a frequency sweep is initiated to determine the desired treatment waveform 220. The desired treatment waveform may also be considered an optimal treatment waveform as determined by the operator. However, in at least some embodiments, the desired treatment waveform may be a composite waveform having at least two component signals. A first of the two component signals may be higher or lower than the second of the two component signals. Accordingly, the operator will apply the waveform and modulate the applied frequency or frequencies while monitoring for the impedance within the patient's tissue. In at least one embodiment, the first component signal may be determined by identifying a frequency at which impedance in the patient's tissue is minimized within the range of approximately 1 Hz to 30 KHz. In at least one embodiment, the second component signal may be determined by identifying a frequency at which impedance in the patient's tissue is maximized, within the range of approximately 1 Hz to 3000 Hz, and when applied as a composite waveform with the first component signal.

Once the desired treatment waveform is determined, the electrode may be moved to various locations in the treatment region to locate at least a first neurologically disrupted area 230 for treatment. In doing so, the technician will monitor the patient's reaction for compensation patterns, along with tissue behavior and various parameters of the waveform as reported by the feedback loop current circuit 170 in order to identify a neurologically disrupted area. In a basic embodiment of the methodology, treatment may begin once a first disrupted area is identified, however, in a preferred embodiment, at least one other neurologically disrupted area is located prior to treatment as depicted at step 240.

In a most preferred embodiment, the depicted method also calls for identification of auxiliary treatment points 250. Auxiliary treatment points may include symmetrical points on the opposite side of the body as the neurologically disrupted area. By way of non-limiting example, if the neurologically disrupted area is on or around the right knee, then an auxiliary treatment point will include the same location on the left knee.

The method may then call for concurrently treating the first and second neurologically disrupted areas 260 as well as concurrently treating the first and second neurologically disrupted areas along with the auxiliary treatment points 270. Treatment may include directing the patient to move the affected area through a variety of routines while applying the desired treatment waveform.

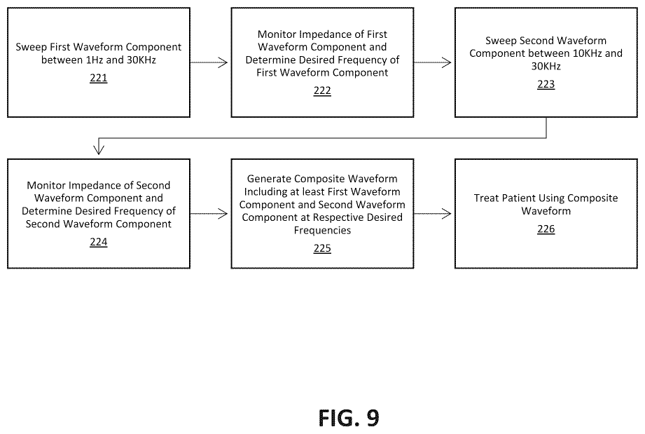

Now turning to FIG. 9, depicted therein is a method for conducting a frequency sweep according to one method of the present invention. Once electrodes have been applied to the patient, the method begins by applying a first waveform component to the patient, and sweeping the first waveform component in a range including 1 Hz and 30 KHz 221. During the sweep, the operator will monitor the impedance of the first waveform component in the patient's tissue and determine a desired frequency of the first waveform component 222. In a most preferred embodiment, the desired frequency is the frequency at which the impedance is lowest. The sweep and monitor technique is then repeated for a second waveform component, as shown at 223 and 224, except that the frequency range of the sweep includes the range 1 Hz to 3000 Hz. In a most preferred embodiment, the desired frequency of the second waveform component may include that frequency at which impedance in the patient's tissue is highest when the first and second waveform components are applied as a composite waveform. Next, the apparatus may be set to generate a composite waveform that includes at least the first waveform component and the second waveform component at their respective desired frequencies 225. Finally, the patient may be treated using the composite waveform 226.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method of treating a patient comprising:
applying an initial scanning waveform to a patient's skin at a first area associated with a site of neurological disruption;
monitoring at least one characteristic of said initial scanning waveform to determine whether said first area comprises a first neurologically disrupted site;
upon locating said first neurologically disrupted site, scanning for at least a second neurologically disrupted site;
initiating a frequency sweep to determine desired treatment frequencies;
locating auxiliary treatment points;
treating at least said first and said second neurologically disrupted sites concurrent to treatment of said auxiliary treatment points.

2. The method as recited in claim 1 wherein said step of initiating a frequency sweep comprises the steps of:
sweeping a first waveform component in a frequency range that includes 1 Hz and 30 KHz;
monitoring impedance of said first waveform component to determine a desired frequency of said first waveform component;
sweeping a second waveform component in a frequency range that includes 1 Hz and 3000 Hz;
monitoring impedance of said second waveform component to determine a desired frequency of said second waveform component.

3. The method as recited in claim 2 wherein said desired frequency of said first waveform component is at least partially defined as a frequency at which minimum impedance occurs.

4. The method as recited in claim 2 wherein said desired frequency of said second waveform component is at least partially defined as a frequency at which maximum impedance occurs.

5. The method as recited in claim 2 further comprising the step of applying said desired frequency of said first waveform component while sweeping said second waveform component.

6. The method as recited in claim 1 wherein said auxiliary treatment points include regions on the patient's body that are oppositely located to either of the first or second neurologically disrupted site.

7. The method as recited in claim 1 further comprising the step of concurrently treating said first and said second neurologically disrupted sites.

8. The method as recited in claim 1 further comprising the step of concurrently treating said first and said second neurologically disrupted sites along with said auxiliary treatment points.

9. A method of treating a patient comprising:
applying an initial waveform to the patient via at least one electrode;
sweeping said initial waveform through a frequency range that includes 1 Hz and 30 KHz, and monitoring parameters of said waveform to determine a frequency having the lowest impedance in the patient's tissue;
applying at least a second waveform to the patient via the at least one electrode, sweeping said second waveform through a frequency range that includes 1 Hz and 3000 Hz, and monitoring parameters of said second waveform to determine a frequency having the highest impedance in the patient's tissue;
scanning for at least one site of neurological disruption;
treating the patient by causing the patient to move the at least one neurologically disrupted site through one or more predetermined routines while applying at least one of said initial and said second waveform via said at least one electrode.

10. The method as recited in claim 9 further comprising the step of scanning for at least a second site of neurological disruption.

11. The method as recited in claim 10 further comprising the step of concurrently treating said first and said second sites of neurological disruption.

12. The method as recited in claim 11 further comprising the step of locating at least one auxiliary treatment point.

13. The method as recited in claim 12 further comprising the step of concurrently treating said first and said second sites of neurological disruption along with said auxiliary treatment point.

14. The method as recited in claim 12 wherein said auxiliary treatment point is located oppositely on the patient's body to either said first or said second sites of neurological disruption.
